SEQUENCE LISTING (12 sequences)

SEQ ID NO: 1 - DNA, 180 bp, Homo sapiens
aggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctcccca

SEQ ID NO: 2 - protein, 60 aa, Homo sapiens
RKVKVLGSGAFGTVYKGIWIPDGENVKIPVAIKVLRENTSPKANKEILDEAYVMAGVGSP

SEQ ID NO: 3 - DNA, 57 bp, Homo sapiens
gaaatcttagacgaagcatacgtgatggcatacgtgatggctggtgtgggctcccca

SEQ ID NO: 4 - protein, 19 aa, Homo sapiens
EILDEAYVMAYVMAGVGSP

SEQ ID NO: 5 - DNA, 54 bp, Homo sapiens
gaaatcttagacgaagcatacgtgatggctggtgtgggctctgtgggctcccca

SEQ ID NO: 6 - protein, 18 aa, Homo sapiens
EILDEAYVMAGVGSVGSP

SEQ ID NO: 7 - protein, 571 aa, Homo sapiens
PTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 8 - protein, 467 aa, Homo sapiens
GNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 9 - protein, 569 aa, Homo sapiens
KIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 10 - protein, 600 aa, Homo sapiens
SAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 11 - DNA, 12 bp, Homo sapiens
gcatacgtgatg

SEQ ID NO: 12 - protein, 4 aa, Homo sapiens
AYVM

(19) United States
(12) Patent Application Publication
(10) Pub. No.: US 2008/0206248 A1
(43) Pub. Date: Aug. 28, 2008

(54) Mutations in ErbB2 associated with cancerous phenotypes

(76) Inventors: Michael Stratton, Hinxton (GB); Andrew Futreal, Hinxton (GB); Richard Wooster, Hinxton (GB)

Correspondence Address: FROMMER LAWRENCE & HAUG, 745 FIFTH AVENUE- 10TH FL., NEW YORK, NY, 10151, US

(21) Appl. No.: 11/699,214

(22) Filed: Jan. 29, 2007

Related U.S. Application Data
(63) Continuation-in-part of application No. PCT/GB05/02976, filed on Jul. 29, 2005.

(30) Foreign Application Priority Data
Jul. 30, 2004 (GB) 0417107.0

(51) Int. Cl. A61K 39/395; C12N 9/12; C07H 21/04; C07K 16/40; C12Q 1/68; G01N 33/53

(52) U.S. Cl. 424/138.1; 435/194; 536/23.1; 536/24.5; 536/24.33; 530/387.7; 435/6; 435/7.1; 435/7.72; 702/19

(57) ABSTRACT

The invention relates to mutations in ErbB2 gene products. The mutations described are identified in human tumours of natural origin. These mutations are associated with cancerous phenotypes and can be used as a basis for the diagnosis of cancer, cancerous cells or a predisposition to cancer in human subjects, selection of appropriate anti-cancer therapy and the development of anti-cancer therapeutics.

This application is a continuation-in-part of International Patent Application PCT/GB2005/002976 filed Jul. 29, 2005 and published as WO 2006/010938 on Feb. 2, 2006, which claims priority from Great Britain Patent Application No. 0417107.0 filed Jul. 30, 2004, and from U.S. Patent Application No. 60/592,538 filed Jul. 30, 2004.

Each of the above referenced applications, and each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

### FIELD OF THE INVENTION

The present invention relates to cancer-specific mutants of the ErbB2/Her2 gene (neu) and uses thereof in the detection of abnormal cells and cancer. Moreover, the invention describes methods for the diagnosis of cancer, the detection of cancerous cells in subjects and the development of therapeutic agents for the treatment of cancer.

### INTRODUCTION

Cancer can develop in any tissue of any organ at any age. Most cancers detected at an early stage are potentially curable; thus, the ability to screen patients for early signs of cancer, and thus allowing for early intervention, is highly desirable (See, for instance, the Merck Manual of Diagnosis and Therapy (1992) 16th ed., Merck & Co).

Moreover, different cancers respond differently to therapy designed to treat them. Since all cancers arise from mutations in genes involved in cell proliferation, differentiation and death, anticancer therapy has been designed to target the products of these genes to potentiate or (usually) inhibit their activity. Modulators of tumourigenic gene products must be specific if they are to be therapeutically useful, so it is important to understand which gene product must be targeted in a particular cancer in order to administer the correct therapy.

Cancerous cells display unregulated growth, lack of differentiation, and ability to invade local tissues and metastasise. Thus cancer cells are unlike normal cells, and are potentially identifiable by not only their phenotypic traits, but also by their biochemical and molecular biological characteristics. Such characteristics are in turn dictated by changes in cancerous cells which occur at the genetic level in a subset of cellular genes known as oncogenes, which directly or indirectly control cell growth and differentiation.

The ErbB2, Her2 and neu gene products were originally identified as separate oncogenes and subsequently shown to be identical. ErbB2/Her2/neu (hereafter: ErbB2) is a protein tyrosine kinase closely related to epidermal growth factor (EGFR; also known as ErbB1/Her1), Her3/ErbB3 and Her4/ErbB4.

Initial observations of ErbB2 involvement in cancer indicated that overexpression of ErbB2 was involved in many human cancers and is associated with a poor prognosis. For example, Semba et al. Proc. Nat. Acad. Sci. 82: 6497-6501 (1985) observed about 30-fold amplification of ErbB2 in a human adenocarcinoma of the salivary gland; Fukushige et al., Molec. Cell. Biol. 6: 955-958 (1986) observed amplification and elevated expression of the ErbB2 gene in a gastric cancer cell line; Di Fiore et al. Science 237: 178-182 (1987) demonstrated that overexpression alone can convert the gene for a normal growth factor receptor, namely, ErbB2, into an oncogene; Van de Vijver et al. New Eng. J. Med. 319: 1239-1245 (1988) found a correlation between overexpression of NEU protein and ductal carcinoma; and Slamon et al. Science 244: 707-712 (1989) described the role of BER2/NEU in breast and ovarian cancer, which together account for one-third of all cancers in women and approximately one-quarter of cancer-related deaths in females.

Overexpression of ErbB2 confers moreover Taxol resistance in breast cancers. Yu et al. Molec. Cell 2: 581-591 (1998) found that overexpression of ErbB2 inhibits Taxol-induced apoptosis. Taxol activates CDC2 kinase in MDA-MB-435 breast cancer cells, leading to cell cycle arrest at the G2/M phase and, subsequently, apoptosis. It appears that ErbB2 can confer resistance to taxol-induced apoptosis by directly phosphorylating CDC2.

The ErbB2 gene is amplified and ErbB2 is overexpressed in 25 to 30% of breast cancers, increasing the aggressiveness of the tumour. Slamon et al., New Eng. J. Med. 344: 783-792 (2001), found that herceptin, a monoclonal antibody specific for the ErbB2 gene product, increased the clinical benefit of first-line chemotherapy in metastatic breast cancer that overexpresses ErbB2.

More recent work confirms that overexpression of ErbB2 is correlated with cancer. For instance, Bhattacharya et al., (2003) BBRC 307:267-273 confirm that “overexpression of ErbB2 is frequent in breast cancer and has been linked to a poor prognosis”. The same is true in lung cancer, where for example Hisch and Langer, (2004) Seminars in Oncology 31, Suppl 1:75-82 confirm that ErbB2 is overexpressed in 16% to 57% of patients with NSCLC (non-small cell lung cancer).

Recently, mutations in EGFR have been identified which show correlation between the effectiveness of anti-EGFR anticancer drugs and clinical outcome, which had previously been elusive (Paez et al., (2004) Science 304:1497-1500; Lynch et al., (2004) New Engl. J. Med. 350:2129-2139; reviewed in Stratton and Futreal, (2004) Nature 430:30.

From these studies, it appears that most cancers which respond to the EGFR inhibitor gefitinib have mutations in EGFR, which was not previously assumed to be required for tumorigenesis. The mutations observed, all in the catalytic kinase domain of EGFR, were point substitutions (G719C, L858R, L861Q) or deletions (delE746-A750, delL747-T751insS, delL747-P753insS). These mutations were observed in 14 out of 15 patients responding to gefitinib treatment, compared to 0 out of 7 in non-responding patients.

A known mechanism for the conversion of proto-oncogenes to oncogenes is the appearance of single mutations in the DNA sequence, known as point mutations, which result in a change in the amino acid sequence of the encoded polypeptide. For example, ras oncogenes are not present in normal cells, but their proto-oncogene counterparts are present in all cells. The wild-type Ras proteins are small GTP-binding proteins that are involved in signal transduction. However, many ras oncogenes from viruses and human tumours have a point mutation in codon number 12: the codon GGC that normally encodes a glycine is changed to GTC, which encodes a valine. Multiple mutations have been documented at this codon, including at least 5 different substitutions which are activating. This single amino acid change prevents the GTPase activity of the Ras protein, and renders Ras constitutively activated, since it remains GTP-bound. The amino acids at positions 13 and 61 are also frequently changed in ras oncogenes from human tumours. Mutations have previously not been shown to be associated with the activity of ErbB2 in lung cancer.

### SUMMARY OF THE INVENTION

Mutations in ErbB2 genes and gene products are described herein. The mutations described are identified in human tumours of natural origin. These mutations are associated with cancerous phenotypes and can be used as a basis for the diagnosis of cancer, cancerous cells or a predisposition to cancer in human subjects, and for the prediction of the efficacy of anti-ErbB2 therapy in cancer patients. Unlike the mutations described in EGFR, the mutations according to the invention comprise insertion mutations as well as point mutations (substitutions).

In a first aspect of the invention, therefore, there is provided a naturally occurring cancer-associated mutant of a human ErbB2 polypeptide, comprising one or more mutations.

Mutant ErbB2 is found to be associated with a number of tumours, including glioma, gastric tumours, and especially NSCLC adenocarcinomas. Preferably, the mutant polypeptide which is associated with NSCLC and isolatable from patients presenting with NSCLC. The mutation is advantageously in the kinase domain of ErbB2.

Surprisingly, it has been found that the responsiveness of cancers to anti-ErbB2 therapy is dependent on the presence of a mutated ErbB2 gene in the patient. It is not sufficient, as has been postulated in the prior art, for the expression of ErbB2 to be overexpressed. It is believed that tumours which express mutated ErbB2 are ErbB2 dependent, and that these tumours are the tumours which respond to therapy which targets the activity of ErbB2. In contrast, tumours in which ErbB2 is overexpressed in a wild-type form do not seem to respond to anti-ErbB2 therapy.

Thus, the identification, for the first time, of mutants of ErbB2 in association with disease and in correlation with a therapeutic approach allows the diagnosis of tumours as responsive to anti-ErbB2 therapy or otherwise, and the more successful selection of therapy for tumours.

Mutations according to the invention may be insertions, deletions or substitutions of amino acids. Preferably, however, the mutation is an insertion, which duplicates a particular string of amino acids, or a point substitution. Point substitutions may comprise substitution of one or more, for example 2 adjacent, amino acids, or 3, 4, 5 or 6 adjacent amino acids.

Preferably, the insertion occurs at position 774 or 779 and is advantageously selected from the group consisting of ins774(AYVM) and ins779(VGS).

Preferably, the amino acid substitution occurs at any one of positions 755, 914 and 776. Advantageously, the amino acid substitution is selected from the group consisting of L755P, E914K and G776S.

Moreover, there is provided a fragment of an ErbB2 polypeptide according to the invention, wherein said fragment comprises the mutation as described above.

In a second aspect, there is provided a nucleic acid encoding a polypeptide according to the first aspect of the invention, or a nucleic acid complementary thereto. In particular, the invention provides the complement of a nucleic acid selected from the group consisting of:

a nucleic acid encoding an ErbB2 polypeptide according to the first aspect of the invention; a nucleic acid encoding an ErbB2 polypeptide according to the first aspect of the invention, wherein the nucleic acid comprises one or more point mutations; a nucleic acid encoding an ErbB2 polypeptide according to the first aspect of the invention, wherein the nucleic acid comprises one or more insertions; a nucleic acid encoding an ErbB2 polypeptide according to the first aspect of the invention which comprises one or more point mutations, wherein the point mutation occurs at one or more of positions 2263, 2704 and 2326 of ErbB2; a nucleic acid encoding an ErbB2 polypeptide according to the first aspect of the invention, which comprises one or more point mutations, wherein the point mutation is HetTT2263/4CC, HetG2740A or HetG2326A; a nucleic acid encoding an ErbB2 polypeptide according to the first aspect of the invention which comprises one or more insertions, wherein the insertion occurs at one or more of positions 2322 or 2335 of ErbB2; and a nucleic acid encoding an ErbB2 polypeptide according to the first aspect of the invention, which comprises one or more insertions, wherein the insertion is Het2322dup12 nt or Het2335ins9 nt.

In a further embodiment, the invention provides nucleic acid which hybridises specifically to a nucleic acid selected as described above. Such a nucleic acid can, for example, be a primer which directs specific amplification of a nucleic acid as described above.

According to a third aspect, there is provided a ligand which binds selectively to a polypeptide according to the first aspect of the invention. Preferably the ligand is an immunoglobulin, for example an antibody or an antigen-binding fragment thereof.

The mutations identified herein are somatic mutations, that is they are not transmitted through the germ line. Accordingly, in a fourth aspect, there is provided a method for the detection of oncogenic mutations, comprising the steps of:
(a) isolating a sample of naturally-occurring cellular material from a human subject;
(b) examining nucleic acid material from at least part of one or more ErbB2 genes in said cellular material; and
(c) determining whether such nucleic acid material comprises one or more mutations in a sequence encoding an ErbB2 polypeptide.

Preferably, the method comprises the steps of:
(a) isolating a first sample of cellular material from a naturally-occurring tissue of a subject which is suspected to be cancerous, and a second sample of cellular material from a non-cancerous tissue of the same subject;
(b) examining nucleic acid material from at least part of one or more ErbB2 genes in both said samples of cellular material; and
(c) determining whether such nucleic acid material comprises one or more mutations in a sequence encoding an ErbB2 polypeptide; and said mutation being present in the naturally-occurring cellular material from the suspected cancerous tissue but not present in the cellular material from the non-cancerous tissue.

Advantageously, the mutation is as described above.

In a fifth aspect, there is provided a method for the detection of oncogenic mutations, comprising the steps of:
(a) obtaining a sample of cellular material from a subject;
(b) screening said sample with a ligand according to the invention; and
(c) detecting one or more mutant ErbB2 polypeptides in said sample.

Advantageously, the mutant ErbB2 polypeptide is a polypeptide according to the first aspect of the invention.

Automated methods, apparata and assays for detection of mutants according to the invention are also provided.

Four separate insertion mutations have been identified in clinical samples to date, as set forth in Table 1 below. This indicates an overall prevalence of at least 4.2% ( 5/120) in unselected primary NSCLC. The frequency in adenocarcinoma subtype of NSCLC is at least 5/51 (9.8%).

To emphasise the relevance of these findings, the frequency of ErbB2 mutations is more than twice that recently reported for EGFR mutations in an unselected series of NSCLC by Paez et al. (Science, (2004) 304(5676):1497-500).

These findings have immediate therapeutic and diagnostic implications. An ErbB2 directed therapeutic (trastuzumab/Herceptin®) has been approved for treatment of metastatic breast cancer and is under evaluation for use in NSCLC. The identification NSCLC patients with ErbB2 mutations may provide a very significant tool for patient stratification in more rational trial designs and diagnostic targeting of those patients with what may be the most responsive tumours. Other monoclonal antibodies which target ErbB2 are in development, such as Omnitarg® (Pertuzumab), which impedes ErbB2 dimerisation. Moreover a selective ErbB2 small molecule inhibitor has been reported (Biochem Biophys Res Commun. 2003 Jul. 25; 307(2):267-73; US Patent Application Publication 2003/0171386) that may be of interest in patients with ErbB2 mutant tumours. Likewise inhibitors with equipotency for both EGFR and ErbB2 may be of interest (Cancer Res. 2001 Oct. 1; 61(19):7196-203 and Bioorg Med Chem. Lett. 2003 Feb. 24; 13(4):637-40).

Accordingly, the invention provides a method for determining whether a patient is expected to be responsive to anti-ErbB2 therapy, comprising the steps of:
(a) isolating a sample of naturally-occurring cellular material from a human subject;
(b) examining nucleic acid material from at least part of one or more ErbB2 genes in said cellular material; and
(c) determining whether such nucleic acid material comprises one or more mutations in a sequence encoding an ErbB2 polypeptide.

The method may also be practised at the polypeptide level, in which case it advantageously comprises the steps of
(a) obtaining a sample of cellular material from a subject;
(b) screening said sample with a ligand according to the invention; and
(c) detecting one or more mutant ErbB2 polypeptides in said sample.

In a preferred embodiment, the invention provides a method for treating a patient suffering from a tumour, comprising the steps of:
(a) determining if the tumour is ErbB2-dependent; and
(b) treating patients having ErbB2 dependent tumours with an inhibitor of ErbB2 activity.

As provided by the present invention, ErbB2 dependency can be determined by observing a mutation in a ErbB2. Advantageously, the mutation is a mutation as set forth above. Preferred inhibitors of ErbB2 activity include Herceptin®, Omnitarg® and small molecule ErbB2 inhibitors, for example inhibitors as set forth in US Patent Application Publication 2003/0171386.

Accordingly, the invention further provides a method for determining whether a patient is susceptible to therapy with Herceptin® or Onnitarg®, comprising the steps of:
(a) determining whether the patient is suffering from an ErbB2 dependent tumour; and
(b) administering Herceptin® and/or Omnitarg® to patients suffering from ErbB2 dependent tumours.

### BRIEF DESCRIPTION OF THE FIGURES

FIG. 1 Shows a partial sequence of ErbB2, indicating the location and nature of some of the insertion mutations observed.

FIG. 2 is a CLUSTAL W (1.82) sequence alignment of EGFR, ErbB2, KIT and PDGFRA. Highlighted regions indicate the position and amino acids affected by mutations.

PDGFRA and EGFR are in-frame deletions, EFGR missense in grey
KIT—both in-frame deletions and insertions
ERBB2—in-frame insertions plus missense in underlined in purple
The position of the G-loop, AIK motif, Catalytic loop and DFG of the activation segment are boxed in yellow for orientation.

FIG. 3 shows a series of tests for transforming activity of ErbB2 mutants in cell-based assays.

### DETAILED DESCRIPTION OF THE INVENTION

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

### DEFINITIONS

The present application describes ErbB2 polypeptide mutants. As used herein, the term “ErbB2 polypeptide” is used to denote a polypeptide encoded by ErbB2/Her2/neu. The term “ErbB2” thus encompasses all known human ErbB2 homologues and variants, as well as other polypeptides which show sufficient homology to ErbB2 to be identified as ErbB2 homologues. The tem does not include EGFR, Her3 or Her4. Preferably, ErbB2 is identified as a polypeptide having the sequence shown at NCBI accession no. NM_{—}004448.1, GI:4758297.

The term “ErbB2” preferably includes polypeptides which are 85%, 90%, 95%, 96%, 97%, 98% or 99% homologous to NM_{—}004448.1. Homology comparisons can be conducted by eye, or more usually, with the aid of readily available sequence comparison programs. These commercially available computer programs can calculate percentage (%) homology between two or more sequences.

Percentage homology can be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

A “fragment” of a polypeptide in accordance with the invention is a polypeptide fragment which encompasses the mutant amino acid(s) described in accordance with the invention. The fragment can be any length up to the full length of ErbB2 polypeptide; it thus encompasses ErbB2 polypeptides which have been truncated by a few amino acids, as well as shorter fragments. Advantageously, fragments are between about 1250 and about 5 amino acids in length; preferably about 5 to about 20 amino acids in length; advantageously, between about 10 and about 50 amino acids in length. Fragments according to the invention are useful, inter alia, for immunisation of animals to raise antibodies. Thus, fragments of polypeptides according to the invention advantageously comprise at least one antigenic determinant (epitope) characteristic of mutant ErbB2 as described herein. Whether a particular polypeptide fragment retains such antigenic properties can readily be determined by routine methods known in the art. Peptides composed of as few as six amino acid residues ore often found to evoke an immune response.

A “nucleic acid” of the present invention is a nucleic acid which encodes a human ErbB2 polypeptide as described above. The term moreover includes those polynucleotides capable of hybridising, under stringent hybridisation conditions, to the naturally occurring nucleic acids identified above, or the complement thereof. “Stringent hybridisation conditions” refers to an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulphate, and 20 pg/ml denatured, sheared salmon sperm DNA, followed by washing the filters in 0.1×SSC at about 65° C.

Although nucleic acids, as referred to herein, are generally natural nucleic acids found in nature, the term can include within its scope modified, artificial nucleic acids having modified backbones or bases, as are known in the art.

A nucleic acid encoding a fragment according to the invention can be the result of nucleic acid amplification of a specific region of a ErbB2 gene, incorporating a mutation in accordance with the present invention.

An “isolated” polypeptide or nucleic acid, as referred to herein, refers to material removed from its original environment (for example, the natural environment in which it occurs in nature), and thus is altered by the hand of man from its natural state. For example, an isolated polynucleotide could be part of a vector or a composition of matter, or could be contained within a cell, and still be “isolated” because that vector, composition of matter, or particular cell is not the original environment of the polynucleotide. Preferably, the term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polypeptides/nucleic acids of the present invention.

The polypeptides according to the invention comprise one or more mutations. “Mutations” includes amino acid addition, deletion or substitution; advantageously, it refers to amino acid substitutions or insertions in the form of insertions. Such mutations at the polypeptide level are reflected at the nucleic acid level by addition, deletion or substitution of one or more nucleotides. Generally, such mutations do not alter the reading frame of the nucleic acid. Advantageously, the changes at the nucleic acid level are point mutations at one or two adjacent positions, or insertions.

The mutations in ErbB2 identified in the present invention occur naturally, and have not been intentionally induced in cells or tissue by the application of carcinogens or other tumourigenic factors. Thus, the mutations identified herein accurately reflect natural tumorigenesis in human tissues to in vivo. Their detection is thus a far better basis for diagnosis than the detection of mutations identified in rodents after artificial chemical tumour induction.

The mutations identified herein are somatic mutations.

A “somatic” mutation is a mutation which is not transmitted through the germ line of an organism, and occurs in somatic tissues thereof. Advantageously, a somatic mutation is one which is determined to be somatic though normal/tumour paired sample analysis.

All amino acid and nucleotide numbering used herein starts from amino acid +1 of the ErbB2 polypeptide or the first ATG of the nucleotide sequence encoding it.

“Amplification” reactions are nucleic acid reactions which result in specific amplification of target nucleic acids over non-target nucleic acids. The polymerase chain reaction (PCR) is a well known amplification reaction.

An “immunoglobulin” is one of a family of polypeptides which retain the immunoglobulin fold characteristic of immunoglobulin (antibody) molecules, which contains two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention is preferably applicable to antibodies, which are capable of binding to target antigens with high specificity.

“Antibodies” can be whole antibodies, or antigen-binding fragments thereof. For example, the invention includes fragments such as Fv and Fab, as well as Fab′ and F(ab′)₂, and antibody variants such as scFv, single domain antibodies, Dab antibodies and other antigen-binding antibody-based molecules.

“Cancer” is used herein to refer to neoplastic growth arising from cellular transformation to a neoplastic phenotype. Such cellular transformation often involves genetic mutation; in the context of the present invention, transformation involves genetic mutation by alteration of one or more ErbB2 genes as described herein.

### Methods for Detection of Nucleic Acids

The detection of mutant nucleic acids encoding ErbB2 can be employed, in the context of the present invention, to diagnose the presence or predisposition to cellular transformation and cancer. Since mutations in ErbB2 genes generally occur at the DNA level, the methods of the invention can be based on detection of mutations in genomic DNA, as well as transcripts and proteins themselves. It can be desirable to confirm mutations in genomic DNA by analysis of transcripts and/or polypeptides, in order to ensure that the detected mutation is indeed expressed in the subject.

Mutations in genomic nucleic acid are advantageously detected by techniques based on mobility shift in amplified nucleic acid fragments. For instance, Chen et al., Anal Biochem 1996 Jul. 15; 239(1):61-9, describe the detection of single-base mutations by a competitive mobility shift assay. Moreover, assays based on the technique of Marcelino et al., BioTechniques 26(6): 1134-1148 (June 1999) are available commercially.

In a preferred example, capillary heteroduplex analysis may be used to detect the presence of mutations based on mobility shift of duplex nucleic acids in capillary systems as a result of the presence of mismatches.

Generation of nucleic acids for analysis from samples generally requires nucleic acid amplification. Many amplification methods rely on an enzymatic chain reaction (such as a polymerase chain reaction, a ligase chain reaction, or a self-sustained sequence replication) or from the replication of all or part of the vector into which it has been cloned. Preferably, the amplification according to the invention is an exponential amplification, as exhibited by for example the polymerase chain reaction.

Many target and signal amplification methods have been described in the literature, for example, general reviews of these methods in Landegren, U., et al., Science 242:229-237 (1988) and Lewis, R., Genetic Engineering News 10:1, 5455 (1990). These amplification methods can be used in the methods of our invention, and include polymerase chain reaction (PCR), PCR in situ, ligase amplification reaction (LAR), ligase hybridisation, Qbeta bacteriophage replicase, transcription-based amplification system (TAS), genomic amplification with transcript sequencing (GAWTS), nucleic acid sequence-based amplification (NASBA) and in situ hybridisation. Primers suitable for use in various amplification techniques can be prepared according to methods known in the art.

### Polymerase Chain Reaction (PCR)

PCR is a nucleic acid amplification method described inter alia in U.S. Pat. Nos. 4,683,195 and 4,683,202. PCR consists of repeated cycles of DNA polymerase generated primer extension reactions. The target DNA is heat denatured and two oligonucleotides, which bracket the target sequence on opposite strands of the DNA to be amplified, are hybridised. These oligonucleotides become primers for use with DNA polymerase. The DNA is copied by primer extension to make a second copy of both strands. By repeating the cycle of heat denaturation, primer hybridisation and extension, the target DNA can be amplified a million fold or more in about two to four hours. PCR is a molecular biology tool, which must be used in conjunction with a detection technique to determine the results of amplification. An advantage of PCR is that it increases sensitivity by amplifying the amount of target DNA by 1 million to 1 billion fold in approximately 4 hours. PCR can be used to amplify any known nucleic acid in a diagnostic context (Mok et al., (1994), Gynaecologic Oncology, 52: 247-252).

### Self-Sustained Sequence Replication (3SR)

Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Enzymatic degradation of the RNA of the RNA/DNA heteroduplex is used instead of heat denaturation. RNase H and all other enzymes are added to the reaction and all steps occur at the same temperature and without further reagent additions. Following this process, amplifications of 106 to 109 have been achieved in one hour at 42° C.

### Ligation Amplification (LAR/LAS)

Ligation amplification reaction or ligation amplification system uses DNA ligase and four oligonucleotides, two per target strand. This technique is described by Wu, D. Y. and Wallace, R. B. (1989) Genomics 4:560. The oligonucleotides hybridise to adjacent sequences on the target DNA and are joined by the ligase. The reaction is heat denatured and the cycle repeated.

### Oβ Replicase

In this technique, RNA replicase for the bacteriophage Qβ, which replicates single-stranded RNA, is used to amplify the target DNA, as described by Lizardi et al., (1988) Bio/Technology 6:1197. First, the target DNA is hybridised to a primer including a T7 promoter and a Qβ 5′ sequence region. Using this primer, reverse transcriptase generates a cDNA connecting the primer to its 5′ end in the process. These two steps are similar to the TAS protocol. The resulting heteroduplex is heat denatured. Next, a second primer containing a Qβ 3′ sequence region is used to initiate a second round of cDNA synthesis. This results in a double stranded DNA containing both 5′ and 3′ ends of the Qβ bacteriophage as well as an active 17 RNA polymerase binding site. T7 RNA polymerase then transcribes the double-stranded DNA into new RNA, which mimics the Qβ. After extensive washing to remove any unhybridised probe, the new RNA is eluted from the target and replicated by Qβ replicase. The latter reaction creates 107 fold amplification in approximately 20 minutes.

Alternative amplification technology can be exploited in the present invention. For example, rolling circle amplification (Lizardi et al., (1998) Nat Genet. 19:225) is an amplification technology available commercially (RCAT™) which is driven by DNA polymerase and can replicate circular oligonucleotide probes with either linear or geometric kinetics under isothermal conditions.

In the presence of two suitably designed primers, a geometric amplification occurs via DNA strand displacement and hyperbranching to generate 10¹² or more copies of each circle in 1 hour.

If a single primer is used, RCAT generates in a few minutes a linear chain of thousands of tandemly linked DNA copies of a target covalently linked to that target.

A further technique, strand displacement amplification (SDA; Walker et al., (1992) PNAS (USA) 80:392) begins with a specifically defined sequence unique to a specific target. But unlike other techniques which rely on thermal cycling, SDA is an isothermal process that utilises a series of primers, DNA polymerase and a restriction enzyme to exponentially amplify the unique nucleic acid sequence.

SDA comprises both a target generation phase and an exponential amplification phase.

In target generation, double-stranded DNA is heat denatured creating two single-stranded copies. A series of specially manufactured primers combine with DNA polymerase (amplification primers for copying the base sequence and bumper primers for displacing the newly created strands) to form altered targets capable of exponential amplification.

The exponential amplification process begins with altered targets (single-stranded partial DNA strands with restricted enzyme recognition sites) from the target generation phase.

An amplification primer is bound to each strand at its complementary DNA sequence. DNA polymerase then uses the primer to identify a location to extend the primer from its 3′ end, using the altered target as a template for adding individual nucleotides. The extended primer thus forms a double-stranded DNA segment containing a complete restriction enzyme recognition site at each end.

A restriction enzyme is then bound to the double stranded DNA segment at its recognition site. The restriction enzyme dissociates from the recognition site after having cleaved only one strand of the double-sided segment, forming a nick. DNA polymerase recognises the nick and extends the strand from the site, displacing the previously created strand. The recognition site is thus repeatedly nicked and restored by the restriction enzyme and DNA polymerase with continuous displacement of DNA strands containing the target segment.

Each displaced strand is then available to anneal with amplification primers as above. The process continues with repeated nicking, extension and displacement of new DNA strands, resulting in exponential amplification of the original DNA target.

Once the nucleic acid has been amplified, a number of techniques are available for detection of single base pair mutations. One such technique is Single Stranded Conformational Polymorphism (SSCP). SCCP detection is based on the aberrant migration of single stranded mutated DNA compared to reference DNA during electrophoresis. Mutation produces conformational change in single stranded DNA, resulting in mobility shift. Fluorescent SCCP uses fluorescent-labelled primers to aid detection. Reference and mutant DNA are thus amplified using fluorescent labelled primers. The amplified DNA is denatured and snap-cooled to produce single stranded DNA molecules, which are examined by non-denaturing gel electrophoresis.

Chemical mismatch cleavage (CMC) is based on the recognition and cleavage of DNA mismatched base pairs by a combination of hydroxylamine, osmium tetroxide and piperidine. Thus, both reference DNA and mutant DNA are amplified with fluorescent labelled primers. The amplicons are hybridised and then subjected to cleavage using Osmium tetroxide, which binds to an mismatched T base, or Hydroxylamine, which binds base. Cleaved fragments are then detected by electrophoresis.

Techniques based on restriction fragment polymorphisms (RFLPs) can also be used. Although many single nucleotide polymorphisms (SNPs) do not permit conventional RFLP analysis, primer-induced restriction analysis PCR (PIRA-PCR) can be used to introduce restriction sites using PCR primers in a SNP-dependent manner. Primers for PIRA-PCR which introduce suitable restriction sites can be designed by computational analysis, for example as described in Xiaiyi et al., (2001) Bioinformatics 17:838-839.

In an alternative embodiment, the present invention provides for the detection of gene expression at the RNA level. Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

RT-PCR is used to amplify RNA targets. In this process, the reverse transcriptase enzyme is used to convert RNA to complementary DNA (cDNA), which can then be amplified using PCR. This method has proven useful for the detection of RNA viruses. Its application is otherwise as for PCR, described above.

### Methods for Detection of Polypeptides

The invention provides a method wherein a protein encoded a mutant ErbB2 gene is detected. Proteins can be detected by protein gel assay, antibody binding assay, or other detection methods known in the art.

For example, therefore, mutant ErbB2 polypeptides can be detected by differential mobility on protein gels, or by other size analysis techniques such as mass spectrometry, in which the presence of mutant amino acids can be determined according to molecular weight. Peptides derived from mutant ErbB2 polypeptides, in particular, as susceptible to differentiation by size analysis.

Advantageously, the detection means is sequence-specific, such that a particular point mutation can accurately be identified in the mutant ErbB2 polypeptide. For example, polypeptide or RNA molecules can be developed which specifically recognise mutant ErbB2 polypeptides in vivo or in vitro.

For example, RNA aptamers can be produced by SELEX. SELEX is a method for the in vitro evolution of nucleic acid molecules with highly specific binding to target molecules. It is described, for example, in U.S. Pat. Nos. 5,654,151, 5,503,978, 5,567,588 and 5,270,163, as well as PCT publication WO 96/38579, each of which is specifically incorporated herein by reference.

The SELEX method involves selection of nucleic acid aptamers, single-stranded nucleic acids capable of binding to a desired target, from a library of oligonucleotides. Starting from a library of nucleic acids, preferably comprising a segment of randomised sequence, the SELEX method includes steps of contacting the library with the target under conditions favourable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched library of nucleic acids, then reiterating the steps of binding, partitioning, dissociating and amplifying through as many cycles as desired to yield highly specific, high affinity nucleic acid ligands to the target molecule.

SELEX is based on the principle that within a nucleic acid library containing a large number of possible sequences and structures there is a wide range of binding affinities for a given target. A nucleic acid library comprising, for example a 20 nucleotide randomised segment can have 4²⁰ structural possibilities. Those which have the higher affinity constants for the target are considered to be most likely to bind. The process of partitioning, dissociation and amplification generates a second nucleic acid library, enriched for the higher binding affinity candidates. Additional rounds of selection progressively favour the best ligands until the resulting library is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The iterative selection/amplification method is sensitive enough to allow isolation of a single sequence variant in a library containing at least 10¹⁴ sequences. The method could, in principle, be used to sample as many as about 10¹⁸ different nucleic acid species. The nucleic acids of the library preferably include a randomised sequence portion as well as conserved sequences necessary for efficient amplification. Nucleic acid sequence variants can be produced in a number of ways including synthesis of randomised nucleic acid sequences and size selection from randomly cleaved cellular nucleic acids. The variable sequence portion can contain fully or partially random sequence; it can also contain subportions of conserved sequence incorporated with randomised sequence. Sequence variation in test nucleic acids can be introduced or increased by mutagenesis before or during the selection/amplification iterations and by specific modification of cloned aptamers.

### Antibodies

ErbB2 polypeptides or peptides derived therefrom can be used to generate antibodies for use in the present invention. The ErbB2 peptides used preferably comprise an epitope which is specific for a mutant ErbB2 polypeptide in accordance with the invention. Polypeptide fragments which function as epitopes can be produced by any conventional means (see, for example, U.S. Pat. No. 4,631,211) In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50. and, most preferably, between about 15 to about 30 amino acids. Preferred polypeptides comprising immunogenic or antigenic epitopes are at least 25, 30, 35, 40, 45, 50, 55, 60, 65, 70, 75, 80, 85, 90, 95 or 100 amino acid residues in length.

Antibodies can be generated using antigenic epitopes of ErbB2 polypeptides according to the invention by immunising animals, such as rabbits or mice, with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections can be needed, for instance, at intervals of about two weeks, to provide a useful titre of anti-peptide antibody which can be detected, for example, by ELISA assay using free peptide adsorbed to a solid surface. The titre of anti-peptide antibodies in serum from an immunised animal can be increased by selection of anti-peptide antibodies, for instance, by adsorption to the peptide on a solid support and elution of the selected antibodies according to methods well known in the art.

The ErbB2 polypeptides of the present invention, and immunogenic and/or antigenic epitope fragments thereof can be fused to other polypeptide sequences. For example, the polypeptides of the present invention can be fused with immunoglobulin domains. Chimeric proteins consisting of the first two domains of the human CD4-polypeptide and various domains of the constant regions of the heavy or light chains of mammalian immunoglobulins have been shown to possess advantageous properties in vivo (see, for example, EP 0394827; Traunecker et al., (1988) Nature, 331: 84-86). Enhanced delivery of an antigen across the epithelial barrier to the immune system has been demonstrated for antigens (such as insulin) conjugated to an FcRn binding partner such as IgG or Fc fragments (see, for example, WO 96/22024 and WO 99/04813).

Moreover, the polypeptides of the present invention can be fused to marker sequences, such as a peptide which facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824 (1989), for instance, hexa-histidine provides for convenient purification of the fusion protein. Another peptide tag useful for purification, the “HA” tag, corresponds to an epitope derived from the influenza hemagglutinin protein (Wilson et al., (1984) Cell 37: 767. Thus, any of these above fusions can be engineered using the nucleic acids or the polypeptides of the present invention.

In a preferred embodiment, the invention provides antibodies which specifically recognise ErbB2 mutants as described herein.

Antibodies as described herein are especially indicated for diagnostic applications. Accordingly, they can be altered antibodies comprising an effector protein such as a label. Especially preferred are labels which allow the imaging of the distribution of the antibody in vivo. Such labels can be radioactive labels or radioopaque labels, such as metal particles, which are readily visualisable within the body of a patient. Moreover, they can be fluorescent labels or other labels which are visualisable on tissue

Recombinant DNA technology can be used to improve the antibodies of the invention. Thus, chimeric antibodies can be constructed in order to decrease the immunogenicity thereof in diagnostic or therapeutic applications. Moreover, immunogenicity can be minimised by humanising the antibodies by CDR grafting [see European Patent Application 0 239 400 (Winter)] and, optionally, framework modification [EP 0 239 400; Riechmann, L. et al., Nature, 332, 323-327, 1988; Verhoeyen M. et al., Science, 239, 1534-1536, 1988; Kettleborough, C. A. et al., Protein Engng., 4, 773-783, 1991; Maeda, H. et al., Human Antibodies and Hybridoma, 2, 124-134, 1991; Gorman S. D. et al., Proc. Natl. Acad. Sci. USA, 88, 4181-4185, 1991; Tempest P. R. et al., Bio/Technology, 9, 266-271, 1991; Co, M. S. et al., Proc. Natl. Acad. Sci. USA, 88, 2869-2873, 1991; Carter, P. et al., Proc. Natl. Acad. Sci. USA, 89, 4285-4289, 1992; Co, M. S. et al., J. Immunol., 148, 1149-1154, 1992; and, Sato, K. et al., Cancer Res., 53, 851-856, 1993].

Antibodies as described herein can be produced in cell culture. Recombinant DNA technology can be used to produce the antibodies according to established procedure, in bacterial or preferably mammalian cell culture. The selected cell culture system optionally secretes the antibody product, although antibody products can be isolated from non-secreting cells.

Therefore, the present invention includes a process for the production of an antibody according to the invention comprising culturing a host, e.g. E. coli, an insect cell or a mammalian cell, which has been transformed with a hybrid vector comprising an expression cassette comprising a promoter operably linked to a first DNA sequence encoding a signal peptide linked in the proper reading frame to a second DNA sequence encoding said antibody protein, and isolating said protein.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by an enzyme immunoassay, e.g. a sandwich assay or a dot-assay, or a radioimmunoassay.

For isolation of the antibodies, the immunoglobulins in the culture supernatants or in the ascitic fluid can be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with the target antigen, or with Protein-A.

The invention further concerns hybridoma cells secreting the monoclonal antibodies of the invention. The preferred hybridoma cells of the invention are genetically stable, secrete monoclonal antibodies of the invention of the desired specificity and can be activated from deep-frozen cultures by thawing and recloning.

The invention, in a preferred embodiment, relates to the production of anti mutant ErbB2 antibodies. Thus, the invention also concerns a process for the preparation of a hybridoma cell line secreting monoclonal antibodies according to the invention, characterised in that a suitable mammal, for example a Balb/c mouse, is immunised with a one or more PDGF polypeptides or antigenic fragments thereof, or an antigenic carrier containing a mutant ErbB2 polypeptide; antibody-producing cells of the immunised mammal are fused with cells of a suitable myeloma cell line, the hybrid cells obtained in the fusion are cloned, and cell clones secreting the desired antibodies are selected. For example spleen cells of Balb/c mice immunised with mutant ErbB2 are fused with cells of the myeloma cell line PAI or the myeloma cell line Sp2/0-Ag14, the obtained hybrid cells are screened for secretion of the desired antibodies, and positive hybridoma cells are cloned.

Preferred is a process for the preparation of a hybridoma cell line, characterised in that Balb/c mice are immunised by injecting subcutaneously and/or intraperitoneally between 1 and 100 μg mutant ErbB2 and a suitable adjuvant, such as Freund's adjuvant, several times, e.g. four to six times, over several months, e.g. between two and four months, and spleen cells from the immunised mice are taken two to four days after the last injection and fused with cells of the myeloma cell line PAI in the presence of a fusion promoter, preferably polyethylene glycol. Preferably the myeloma cells are fused with a three- to twentyfold excess of spleen cells from the immunised mice in a solution containing about 30% to about 50% polyethylene glycol of a molecular weight around 4000. After the fusion the cells are expanded in suitable culture media as described hereinbefore, supplemented with a selection medium, for example HAT medium, at regular intervals in order to prevent normal myeloma cells from overgrowing the desired hybridoma cells.

The invention also concerns recombinant nucleic acids comprising an insert coding for a heavy chain variable domain and/or for a light chain variable domain of antibodies directed to mutant ErbB2 as described hereinbefore. By definition such DNAs comprise coding single stranded DNAs, double stranded DNAs consisting of said coding DNAs and of complementary DNAs thereto, or these complementary (single stranded) DNAs themselves.

Furthermore, DNA encoding a heavy chain variable domain and/or for a light chain variable domain of antibodies directed to mutant ErbB2 can be enzymatically or chemically synthesised DNA having the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant DNA is also intended to be a silent mutant wherein one or more nucleotides are replaced by other nucleotides with the degenerated sequence. Degenerated sequences are degenerated within the meaning of the genetic code in that an unlimited number of nucleotides are replaced by other nucleotides without resulting in a change of the amino acid sequence originally encoded. Such degenerated sequences can be useful due to their different restriction sites and/or frequency of particular codons which are preferred by the specific host, particularly E. coli, to obtain an optimal expression of the heavy chain murine variable domain and/or a light chain murine variable domain.

In this context, the term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

The invention therefore also concerns recombinant nucleic acids comprising an insert coding for a heavy chain murine variable domain of an anti mutant ErbB2 antibody fused to a human constant domain γ, for example γ1, γ2, γ3 or γ4, preferably γ1 or γ4. Likewise the invention concerns recombinant DNAs comprising an insert coding for a light chain murine variable domain of an anti mutant ErbB2 antibody directed to mutant ErbB2 fused to a human constant domain κ or λ, preferably κ.

In another embodiment the invention pertains to recombinant DNAs coding for a recombinant polypeptide wherein the heavy chain variable domain and the light chain variable domain are linked by way of a spacer group, optionally comprising a signal sequence facilitating the processing of the antibody in the host cell and/or a DNA coding for a peptide facilitating the purification of the antibody and/or a cleavage site and/or a peptide spacer and/or an effector molecule.

Antibodies and antibody fragments according to the invention are useful in diagnosis. Accordingly, the invention provides a composition for diagnosis comprising an antibody according to the invention.

In the case of a diagnostic composition, the antibody is preferably provided together with means for detecting the antibody, which can be enzymatic, fluorescent, radioisotopic or other means. The antibody and the detection means can be provided for simultaneous, simultaneous separate or sequential use, in a diagnostic kit intended for diagnosis.

The antibodies of the invention can be assayed for immunospecific binding by any method known in the art. The immunoassays which can be used include but are not limited to competitive and non-competitive assay systems using techniques such as western blots, radioimmunoassays, ELISA, sandwich imnunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al, eds, 1994, Current Protocols in Molecular Biology, Vol. 1, John Wiley & Sons, Inc., New York, which is incorporated by reference herein in its entirety). Exemplary immunoassays are described briefly below.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), exposing the membrane to a primary antibody (the antibody of interest) diluted in blocking buffer, washing the membrane in washing buffer, exposing the membrane to a secondary antibody (which recognises the primary antibody, e.g., an antihuman antibody) conjugated to an enzymatic substrate (e.g., horseradish peroxidase or alkaline phosphatase) or radioactive molecule (e.g., ³²P or 125) diluted in blocking buffer, washing the membrane in wash buffer, and detecting the presence of the antigen.

ELISAs comprise preparing antigen, coating the well of a 96 well microtitre plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognises the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well.

The binding affinity of an antibody to an antigen and the off-rate of an antibody-antigen interaction can be determined by competitive binding assays. One example of a competitive binding assay is a radioimmunoassay comprising the incubation of labelled antigen (e.g., ³H or ¹²⁵I) with the antibody of interest in the presence of increasing amounts of unlabeled antigen, and the detection of the antibody bound to the labelled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labelled compound (e.g., ³H or ¹²⁵I) in the presence of increasing amounts of an unlabeled second antibody.

### Preparation of Mutant ErbB2 Polypeptides

Mutant ErbB2 polypeptides in accordance with the present invention can be produced by any desired technique, including chemical synthesis, isolation from biological samples and expression of a nucleic acid encoding such a polypeptide. Nucleic acids, in their turn, can be synthesised or isolated from biological sources of mutant ErbB2.

The invention thus relates to vectors encoding a polypeptide according to the invention, or a fragment thereof. The vector can be, for example, a phage, plasmid, viral, or retroviral vector.

Nucleic acids according to the invention can be part of a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The nucleic acid insert is operatively linked to an appropriate promoter, such as the phage lambda PL promoter, the E. coli lac, trp, phoA and tac promoters, the SV40 early and late promoters and promoters of retroviral LTRs. Other suitable promoters are known to those skilled in the art. The expression constructs further contain sites for transcription initiation, termination, and, in the transcribed region, a ribosome binding site for translation. The coding portion of the transcripts expressed by the constructs preferably includes a translation initiating codon at the beginning and a termination codon (UAA, UGA or UAG) appropriately positioned at the end of the polypeptide to be translated.

As indicated, the expression vectors preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris); insect cells such as Drosophila S2 and Spodoptera Sf9 cells; animal cells such as CHO, COS, 293, and Bowes melanoma cells; and plant cells.

Appropriate culture media and conditions for the above-described host cells are known in the art and available commercially.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK2233, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PA0815 (all available from Invitrogen, Carlsbad, Calif.).

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Sambrook et al., referred to above.

A polypeptide according to the invention can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides according to the present invention can also be recovered from biological sources, including bodily fluids, tissues and cells, especially cells derived from tumour tissue or suspected tumour tissues from a subject.

In addition, polypeptides according to the invention can be chemically synthesised using techniques known in the art (for example, see Creighton, 1983, Proteins: Structures and Molecular Principles, W. H. Freeman & Co., N.Y., and Hunkapiller et al., Nature, 310: 105-111 (1984)). For example, a polypeptide corresponding to a fragment of a mutant ErbB2 polypeptide can be synthesised by use of a peptide synthesiser.

### ErbB2 Mutations

Mutations in ErbB2 have been identified in human tumour cells. Table 1 describes the location of these mutations and the tumours in which they were identified. The mutations are in the kinase domain of ErbB2. Most of the mutations can be confirmed as somatic, indicating that a paired normal/tumour sample was tested and the mutation found only in the tumour sample.

TABLE 1
ERBB2 mutations in primary tumours

| | | | |
|---|---|---|---|
| Sample | Tumour/Histology | Nucleotide^{†} | Amino Acid^{†} |

Lung Cancer

| | | | |
|---|---|---|---|
| PD1353a | NSCLC - adenocarcinoma | 2322 ins/dup(GCATACGTGATG) | ins774(AYVM) |
| PD0258a | NSCLC - adenocarcinoma | 2322 ins/dup(GCATACGTGATG) | ins774(AYVM) |
| PD0317a | NSCLC - adenocarcinoma | 2322 ins/dup(GCATACGTGATG) | ins774(AYVM) |
| PD0319a | NSCLC - adenocarcinoma | 2335 ins(CTGTGGGCT) | ins779(VGS) |
| PD0270a | NSCLC - adenocarcinoma | TT2263-4CC | L755P |

Other

| | | | |
|---|---|---|---|
| PD1487a | Glioblastoma | G2740A | E914K |
| PD1403a | Gastric cancer | G2326A | G776S |
| PD0888a | Ovarian cancer | A2570G | N857S |

^{†}Numbering is relative to the A of the ATG/initiating methionine as nucleotide one in NCBI/RefSeq accession NM₋ 004448.1

In addition, the following mutants have been identified in ErbB2 in cancer patients:

| | | | | | |
|---|---|---|---|---|---|
| stCE17-1157 | PD0312a | NSCLC | adenocar- | Het A560G | N187S |
| | | | cinoma | | |
| stCE17-1163 | PD0293a | NSCLC | squamous | Het C1157A | A386D |
| | | | ca | | |
| stCE17-1174 | DV-90 | NSCLC | adenocar- | Het G2524A | V842I |
| | | | cinoma | | |
| stCE17-1379 | PD0318a | NSCLC | adenocar- | Het C3647A | A1216D |
| | | | cinoma | | |

These variants are of unknown significance as they cannot be proven somatic due to lack of normal tissue. However, in a preferred embodiment, the invention moreover comprises the above mutations in ErbB2 as indicated.

### Compound Assays

According to the present invention, mutant ErbB2 is used as a target to identify compounds, for example lead compounds for pharmaceuticals, which are capable of modulating the proliferative activity of mutant ErbB2. Accordingly, the invention relates to an assay and provides a method for identifying a compound or compounds capable, directly or indirectly, of modulating the activity mutant ErbB2, comprising the steps of:
(a) incubating mutant ErbB2 with the compound or compounds to be assessed; and
(b) identifying those compounds which influence the activity of mutant ErbB2.

Mutant ErbB2 is as defined in the context of the present invention.

According to a first embodiment of this aspect invention, the assay is configured to detect polypeptides which bind directly to mutant ErbB2.

The invention therefore provides a method for identifying a modulator cell proliferation, comprising the steps of:
(a) incubating mutant ErbB2 with the compound or compounds to be assessed; and
(b) identifying those compounds which bind to mutant ErbB2.

Preferably, the method further comprises the step of:
(c) assessing the compounds which bind to mutant ErbB2 for the ability to modulate cell viability or cell proliferation in a cell-based assay.

Binding to mutant ErbB2 may be assessed by any technique known to those skilled in the art. Examples of suitable assays include the two hybrid assay system, which measures interactions in vivo, affinity chromatography assays, for example involving binding to polypeptides immobilised on a column, fluorescence assays in which binding of the compound(s) and mutant ErbB2 is associated with a change in fluorescence of one or both partners in a binding pair, and the like. Preferred are assays performed in vivo in cells, such as the two-hybrid assay.

In preferred embodiments, a nucleic acid encoding mutant ErbB2 is ligated into a vector, and introduced into suitable host cells to produce transformed cell lines that express mutant ErbB2. The resulting cell lines can then be produced for reproducible qualitative and/or quantitative analysis of the effect(s) of potential compounds affecting mutant ErbB2 function. Thus mutant ErbB2 expressing cells may be employed for the identification of compounds, particularly low molecular weight compounds, which modulate the function of mutant ErbB2. Thus host cells expressing mutant ErbB2 are useful for drug screening and it is a further object of the present invention to provide a method for identifying compounds which modulate the activity of mutant ErbB2, said method comprising exposing cells containing heterologous DNA encoding mutant ErbB2, wherein said cells produce functional mutant ErbB2, to at least one compound or mixture of compounds or signal whose ability to modulate the activity of said mutant ErbB2 is sought to be determined, and thereafter monitoring said cells for changes caused by said modulation. Such an assay enables the identification of modulators, such as agonists, antagonists and allosteric modulators, of mutant ErbB2. As used herein, a compound or signal that modulates the activity of mutant ErbB2 refers to a compound that alters the activity of mutant ErbB2 in such a way that the activity of mutant ErbB2 on a target thereof is different in the presence of the compound or signal (as compared to the absence of said compound or signal).

Cell-based screening assays can be designed by constructing cell lines in which the expression of a reporter protein, i.e. an easily assayable protein, such as β-galactosidase, chloramphenicol acetyltransferase (CAT) or luciferase, is dependent on the activation of a mutant ErbB2 substrate. For example, a reporter gene encoding one of the above polypeptides may be placed under the control of an response element which is specifically activated by an ErbB2 target. Such an assay enables the detection of compounds that directly modulate mutant ErbB2 function, such as compounds that antagonise phosphorylation of targets mutant ErbB2, or compounds that inhibit or potentiate other cellular functions required for the activity of mutant ErbB2. Cells in which wild-type, non-mutant ErbB2 is present provide suitable controls.

Alternative assay formats include assays which directly assess proliferative responses in a biological system. The constitutive expression of unregulated mutant ErbB2 results in an proliferative phenotype in animal cells. Cell-based systems, such as 3T3 fibroblasts, may be used to assess the activity of potential regulators of mutant ErbB2.

In a further preferred aspect, the invention relates to a method for identifying a lead compound for a pharmaceutical, comprising the steps of:
providing a purified mutant ErbB2 molecule;
incubating the mutant ErbB2 molecule with a substrate known to be phosphorylated by mutant ErbB2 and a test compound or compounds; and
identifying the test compound or compounds capable of modulating the phosphorylation of the substrate.

Optionally, the test compound(s) identified may then be subjected to in vivo testing to determine their effects on a mutant ErbB2 signalling pathway.

As used herein, “mutant ErbB2 activity” may refer to any activity of mutant ErbB2, including its binding activity, but in particular refers to the phosphorylating activity of mutant ErbB2 and/or the ability of mutant ErbB2 to dimerise with itself and/or other members of the ErbB family, such as EGFR, Her3 and Her4. Accordingly, the invention may be configured to detect the phosphorylation of target compounds by mutant ErbB2, and the modulation of this activity by potential therapeutic agents.

Examples of compounds which modulate the phosphorylating activity of mutant ErbB2 include dominant negative mutants of ErbB2 itself Such compounds are able to compete for the target of mutant ErbB2, thus reducing the activity of mutant ErbB2 in a biological or artificial system. Thus, the invention moreover relates to compounds capable of modulating the phosphorylating activity of mutant ErbB2.

Compounds which influence the activity of mutant ErbB2 may be of almost any general description, including low molecular weight compounds, including organic compounds which may be linear, cyclic, polycyclic or a combination thereof, peptides, polypeptides including antibodies, or proteins. In general, as used herein, “peptides”, “polypeptides” and “proteins” are considered equivalent.

Many compounds according to the present invention may be lead compounds useful for drug development. Useful lead compounds are especially antibodies and peptides, and particularly intracellular antibodies expressed within the cell in a gene therapy context, which may be used as models for the development of peptide or low molecular weight therapeutics. In a preferred aspect of the invention, lead compounds and mutant ErbB2 or other target peptides may be co-crystallised in order to facilitate the design of suitable low molecular weight compounds which mimic the interaction observed with the lead compound.

Crystallisation involves the preparation of a crystallisation buffer, for example by mixing a solution of the peptide or peptide complex with a “reservoir buffer”, preferably in a 1:1 ratio, with a lower concentration of the precipitating agent necessary for crystal formation. For crystal formation, the concentration of the precipitating agent is increased, for example by addition of precipitating agent, for example by titration, or by allowing the concentration of precipitating agent to balance by diffusion between the crystallisation buffer and a reservoir buffer. Under suitable conditions such diffusion of precipitating agent occurs along the gradient of precipitating agent, for example from the reservoir buffer having a higher concentration of precipitating agent into the crystallisation buffer having a lower concentration of precipitating agent. Diffusion may be achieved for example by vapour diffusion techniques allowing diffusion in the common gas phase. Known techniques are, for example, vapour diffusion methods, such as the “hanging drop” or the “sitting drop” method. In the vapour diffusion method a drop of crystallisation buffer containing the protein is hanging above or sitting beside a much larger pool of reservoir buffer. Alternatively, the balancing of the precipitating agent can be achieved through a semipermeable membrane that separates the crystallisation buffer from the reservoir buffer and prevents dilution of the protein into the reservoir buffer.

In the crystallisation buffer the peptide or peptide/binding partner complex preferably has a concentration of up to 30 mg/ml, preferably from about 2 mg/ml to about 4 mg/ml.

Formation of crystals can be achieved under various conditions which are essentially determined by the following parameters: pH, presence of salts and additives, precipitating agent, protein concentration and temperature. The pH may range from about 4.0 to 9.0. The concentration and type of buffer is rather unimportant, and therefore variable, e.g. in dependence with the desired pH. Suitable buffer systems include phosphate, acetate, citrate, Tris, MES and HEPES buffers. Useful salts and additives include e.g. chlorides, sulphates and other salts known to those skilled in the art. The buffer contains a precipitating agent selected from the group consisting of a water miscible organic solvent, preferably polyethylene glycol having a molecular weight of between 100 and 20000, preferentially between 4000 and 10000, or a suitable salt, such as a sulphates, particularly ammonium sulphate, a chloride, a citrate or a tartarate.

A crystal of a peptide or peptide/binding partner complex according to the invention may be chemically modified, e.g. by heavy atom derivatization. Briefly, such derivatization is achievable by soaking a crystal in a solution containing heavy metal atom salts, or a organometallic compounds, e.g. lead chloride, gold thiomalate, thimerosal or uranyl acetate, which is capable of diffusing through the crystal and binding to the surface of the protein. The location(s) of the bound heavy metal atom(s) can be determined by X-ray diffraction analysis of the soaked crystal, which information may be used e.g. to construct a three-dimensional model of the peptide.

A three-dimensional model is obtainable, for example, from a heavy atom derivative of a crystal and/or from all or part of the structural data provided by the crystallisation. Preferably building of such model involves homology modelling and/or molecular replacement.

The preliminary homology model can be created by a combination of sequence alignment with any ErbB/Her protein the structure of which is known, such as ErbB2 itself (Cho et al., Nature 421: 756-760, 2003), secondary structure prediction and screening of structural libraries. For example, the sequences of mutant ErbB2 and a candidate peptide can be aligned using a suitable software program.

Computational software may also be used to predict the secondary structure of the peptide or peptide complex. The peptide sequence may be incorporated into the mutant ErbB2 structure. Structural incoherences, e.g. structural fragments around insertions/deletions can be modelled by screening a structural library for peptides of the desired length and with a suitable conformation. For prediction of the side chain conformation, a side chain rotamer library may be employed.

The final homology model is used to solve the crystal structure of the peptide by molecular replacement using suitable computer software. The homology model is positioned according to the results of molecular replacement, and subjected to further refinement comprising molecular dynamics calculations and modelling of the inhibitor used for crystallisation into the electron density.

### Assays for ErbB2 Activity

The activity of mutant ErbB2 may be assayed, for example, by measuring kinase activity and through cellular transformation assays.

In a first embodiment, mutant forms of the receptor gene are isolated from the tumour to be assessed or constructed from sequence information derived from said tumour. The mutant ErbB2 gene(s) are transiently expressed in cells and then checked for expression by Western blot. The expression of the mutant and wild-type forms is then assayed for its effect on downstream signalling events.

Specifically, activation of the Ras/RAF/MEK/ERK pathway is assayed. This involves performing Ras activation assays using the “Ras-capture” approach (Marais et al., (1998) Science 280(5360):109-12). RAF, MEK and ERK assays are examined using antibodies that recognise the phosphorylated and active forms and also by direct immunoprecipitation kinase activity.

The ERK partway can be assayed as described, for example, in Karasarides M, Chiloeches A, Hayward et al., Oncogene. 2004 Jun 21 [Epub ahead of print]; Wellbrock et al., Cancer Res. 2004 Apr. 1; 64(7):2338-42; or Wan et al., Cell. 2004 Mar. 19; 116(6):855-67.

The assay may optionally be enhanced by examining transcription controls of known genes.

Other pathways that are known to be downstream of EGF signalling, such as the PI3-kinase pathway, may also be exploited for measurement of ErbB2 activity. The approach is similar to the one described above.

In addition, long-term assays based on stable cell lines may be used. Stable lines are created and assessed for transformation by normal criteria in vitro (ability to form colonies, loss of contact inhibition, growth in soft agar) and also for the ability to grow as tumours in nude mice. Finally, more sophisticated assays can be performed, such as testing the ability of the cells to invade matrigel plugs and to migrate in the absence of growth factors.

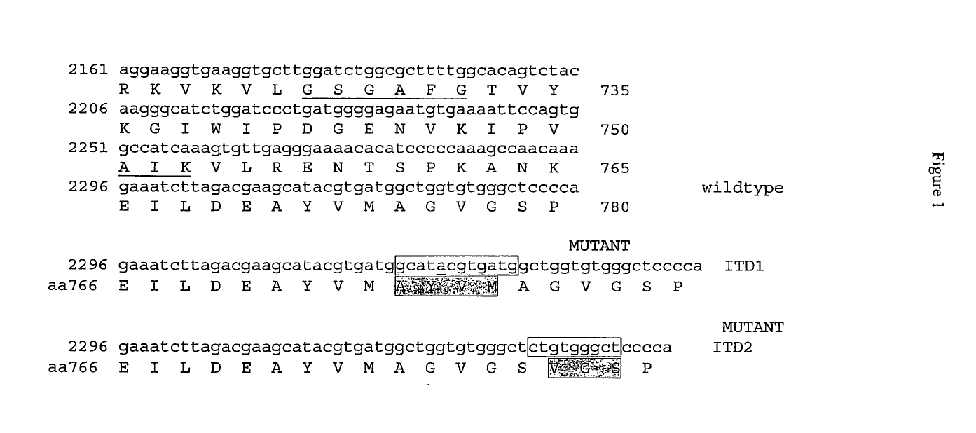

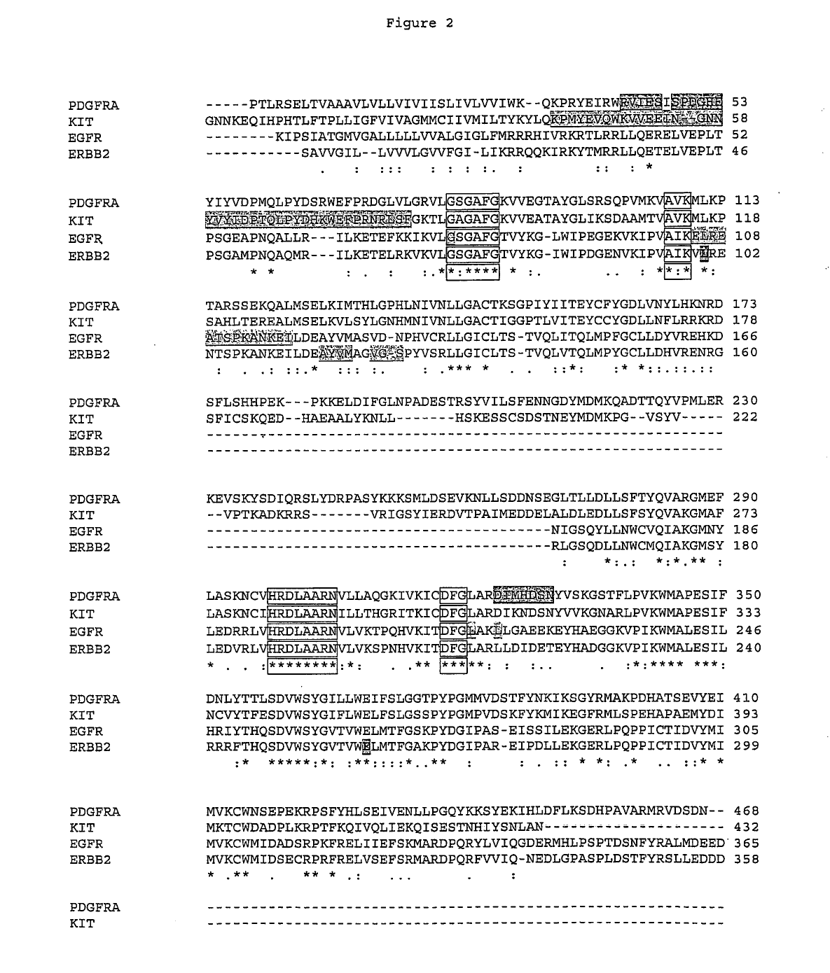

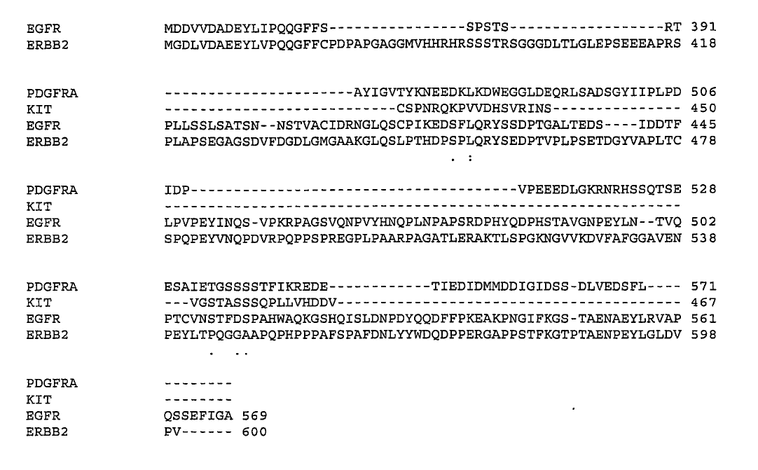

For example, mutant ErbB2 may be transfected in to cell lines using a transfection reagent such as Lipofectamine®. In a example, the plasmid pEF/c-erbB2.6, expressing wild-type ErbB2 under the control of the EF1a promoter, is transfected into NIH3T3 cells. Cells are also transfected with mutants of pEF/c-erbB2.6 comprising the mutations G776S, VGS and VYVM as described herein (see FIG. 1). EGFR mutagenesis is performed using the Quickchange II XL Site-Directed Mutagenesis Kit (Stratagene).

Transfection experiments were performed using lipofectamine reagent (Invitrogen) with NIH3T3 cells in DMEM+5% DCS. 2.5×10⁵ cells per well of a six well dish are plated and incubated overnight.

Transfection complexes are prepared on bacterial culture dishes. Each EGFR (ErbB2) vector is diluted with sterile PBS to 0.016 ug/ul. 0.256 ug of each EGFR vector is used per transfection (Total DNA per well 256 ng). For each transfection, 13 μl of PBS is mixed with 3 μl of lipofectamine on a bacterial plate. 16 μl of the DNA mix is combined with the lipofectamine for 15 minutes at room temperature. While complexes are forming, cells are washed twice with serum free DMEM and 800 ul of serum free DMEM is added to each well. 200 ul of serum free DMEM is added to each complexes (DNA/lipofectamine mix) and the total volume is added to the cells.

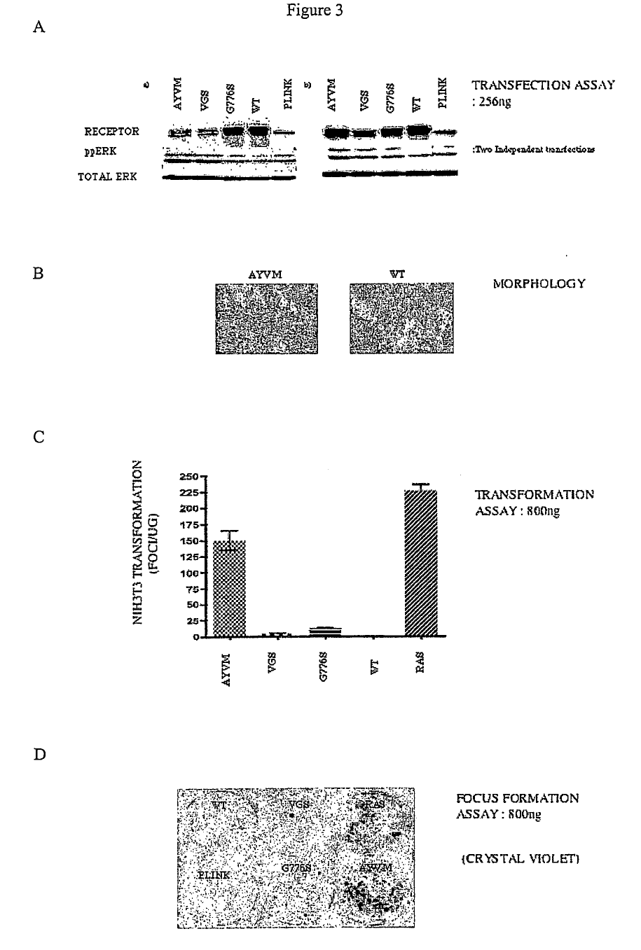

After 6 hours the media are removed, the cells washed twice in DMEM+5% DCS and then 2 ml of DMEM+5% DCS is added. The cells are incubated for 2 days and then harvested by NP40 extraction buffer for western blotting. FIG. 3A shows western blots of two transfection experiments using the above plasmids.

Passage 11 NIH3T3 cells transfected with the plasmids as above, plus a focus formation positive control (Ras) are used in a focus formation assay. Cells are transfected with 800 ng of each EGFR mutant plasmid. The Ras control is transfected at 100 ng together with sufficient plink control vector (no insert in the polylinker) to give a total of 800 ng total DNA.

After 24 hours, cells are trypsinised and split between two 10 cm tissue culture dishes containing 10 ml of DMEM+5% DCS. The media on the cells are changed on day 5, 8, 13 and 15 and the assay is terminated on day 20 by fixing cells in 4% formaldelyde for 30 minutes. FIG. 3B shows the cell morphology observed after 8 days.

To count foci, cells were stained with 4% (w/v) Crystal Violet in 70% ethanol. Only the foci, which were 2 mm in diameter were counted. FIG. 3C shows the results of the focus formation assay; the crystal violet stains are shown in FIG. 3D.

The focus formation assay results were the average of three independent experiments. The mutants according to the invention have potent transforming ability as assessed in vivo in the focus formation assay.

### Computational Aspects of Detection

The detection of mutant ErbB2 polypeptides and/or mutant ErbB2 nucleic acids can be automated to provide rapid massively parallel screening of sample populations. Computerised methods for mutation detection are known in the art, and will generally involve the combination of a sequencing device, or other device capable of detecting sequence variation in polypeptides or nucleic acids, a data processing unit and an output device which is capable of displaying the result in a form interpretable by a technician or physician.

In a preferred aspect, therefore, the invention provides an automated method for detecting a mutation at a target sequence position in a nucleic acid derived from a naturally-occurring primary human tumour encoding a ErbB2 polypeptide, comprising:
sequencing a sample of an amplification product of the nucleic acid from the naturally-occurring primary human tumour to provide a sample data set specifying a plurality of measured base pair identification data in a target domain extending from a start sequence position to an end sequence position;
determining presence or absence of the mutation in the sample conditional on whether the measured base pair identification datum for the target sequence position corresponds to a reference base pair datum for the target sequence position; and
generating an output indicating the presence or absence of the mutation in the sample as established by the determining step.
Methods for sequencing and for detection of mutations in sequences are set forth above and generally known in the art. The invention makes use of such methods in providing an apparatus for carrying out the process of the invention, which apparatus comprises:
a sequence reading device operable to determine the sequence of a sample of a nucleic acid to provide a sample data set specifying measured base pair identification data in a target domain extending from a start sequence position to an end sequence position; and
a data analysis unit connected to receive the sample data set from the sequencing device and operable to determine presence or absence of the mutation in the sample conditional on whether the measured base pair identification datum for the target sequence position corresponds to a reference base pair datum for the target sequence position.

Suitable sequence reading devices include automated sequencers, RFLP-analysers and mobility shift analysis apparata. Advantageously, the sequence of an amplification product of the target nucleic acid is analysed, and the apparatus moreover includes an amplification device such as a PCR machine.

Preferably, the apparatus also comprises an output device operable to generate an output indicating the presence or absence of the mutation in the sample determined by the data analysis unit. For example, the output device can comprise at least one of: a graphical user interface; an audible user interface; a printer; a computer readable storage medium; and a computer interpretable carrier medium.

The invention can moreover be configured to detect the mutant ErbB2 protein itself. Thus, in a further aspect, the invention relates to an automated method for detecting a single amino acid mutation in a ErbB2 polypeptide from a naturally-occurring primary human tumour, comprising:
applying a marker to one or more target amino acids in a sample of the ErbB2 polypeptide;
reading the sample after applying the marker to determine presence or absence of the marker in the sample, thereby to indicate presence or absence of the single amino acid mutation in the sample; and
generating an output indicating the presence or absence of the single amino acid mutation in the sample as determined by the reading step.

The marker preferably comprises a ligand that binds differentially to a wild-type ErbB2 polypeptide without single amino acid mutation and to a mutant ErbB2 polypeptide with the mutation. Preferential binding to either form of ErbB2 is possible in the context of the invention.

The invention moreover provides an apparatus for detecting an amino acid mutation in a ErbB2 polypeptide, comprising:
a protein marking device loaded with a marker and operable to apply a marker to one or more target amino acids in a sample of the ErbB2 polypeptide; and
a marker reading device operable to determine presence or absence of the marker in the sample, thereby to indicate presence or absence of the single amino acid mutation in the sample.

The marker used can be an antibody, and the protein marling device can be configured to implement an ELISA process.

Advantageously, the protein marking device comprises a microarrayer which is preferably configured to read the sample optically.

Preferably, the apparatus comprises an output device operable to generate an output indicating the presence or absence of the single amino acid mutation in the sample as determined by the marker reading device. Suitable output devices comprises at least one of: a graphical user interface; an audible user interface; a printer; a computer readable storage medium; and a computer interpretable carrier medium.

### Uses of the Invention

The present invention provides novel mutants of ErbB2 polypeptides which are useful in the detection of neoplastic conditions, and the determination of prognoses for subjects suffering from such conditions as well as appropriate therapies for such subjects. In general, the presence of a mutation in ErbB2 as described herein is associated with the presence of adenocarcinoma of the lung.

In one aspect, the present invention provides a method for identifying cancerous cells or tissue (such as NSCLC), or of identifying cells or tissue which are predisposed to developing a neoplastic phenotype, comprising: amplifying at least part of an ErbB2 gene of the cells or tissue; analysing the amplification product to detect a mutation in the ErbB2 gene as described herein; wherein a cell or tissue having one or more ErbB2 mutations is categorised as being cancerous or being at an increased risk of developing a cancerous condition. Suitable amplification means include PCR and cloning.

In another embodiment, the present invention relates to a method for determining a therapeutic regime for a subject suffering from NSCLC. The method comprises: amplifying the region of the ErbB2 gene as described above; analysing the amplification products for evidence of mutation as described above; and classifying a subject having no mutations in the ErbB2 gene as being less likely to respond to anti-ErbB2 therapy, and a subject having said mutations as being more likely to respond to anti-ErbB2 therapy.

The techniques according to the invention can be automated, as required for rapid screening of samples for the identification of potentially cancerous conditions. Generally, an automated process will comprise automated amplification of nucleic acid from tissue or cell samples, detection of mutations in amplified nucleic acid, such as by fluorescent detection, and/or displaying the presence of mutations. Exemplary automated embodiments are described above.

The identification of mutant ErbB2 according to the invention can thus be used for diagnostic purposes to detect, diagnose, or monitor diseases, disorders, and/or conditions associated with the expression of mutant ErbB2. In particular, the invention is concerned with the detection, diagnosis and/or monitoring of cancers associated with mutant ErbB2 as set forth herein.

The invention provides a diagnostic assay for diagnosing cancer, comprising (a) assaying the expression of mutant ErbB2 in cells or body fluid of an individual using one or more antibodies specific to the ErbB2 mutant as defined herein. The presence of mutant ErbB2 transcript in biopsy tissue from an individual can indicate a predisposition for the development of the disease, or can provide a means for detecting the disease prior to the appearance of actual clinical symptoms. A more definitive diagnosis of this type allows health professionals to employ appropriate therapies.

Antibodies of the invention can be used to assay protein levels in a biological sample using classical immunohistological methods known to those of skill in the art (e.g., see Jalkanen, et al., (1985) J. Cell. Biol. 101:976-985; Jalkanen, et al., (1987) J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I, carbon (¹⁴C), sulphur (³⁵S), tritium (³H), indium (¹¹²In), and technetium (⁹⁹Tc); luminescent labels, such as luminol; and fluorescent labels, such as fluorescein and rhodamine, and biotin.

Moreover, mutations in ErbB2 can be detected by analysis of nucleic acids, as set forth herein. For example, the presence of mutations can be detected by sequencing, or by SCCP analysis.

The present invention moreover provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody can be conjugated to a detectable substrate such as a fluorescent compound, an enzymatic substrate, a radioactive compound or a luminescent compound, or a second antibody which recognises the first antibody can be conjugated to a detectable substrate).

In another specific embodiment of the present invention, the kit is a diagnostic kit for use in screening serum containing antibodies specific for mutant ErbB2 polypeptides as described herein. Such a kit can include a control antibody that does not react with the mutant ErbB2 polypeptide. Such a kit can include a substantially isolated polypeptide antigen comprising an epitope which is specifically immunoreactive with at least one anti-ErbB2 antibody. Further, such a kit includes means for detecting the binding of said antibody to the antigen (e.g., the antibody can be conjugated to a fluorescent compound such as fluorescein or rhodamine which can be detected by flow cytometry). In specific embodiments, the kit can include a recombinantly produced or chemically synthesised polypeptide antigen. The polypeptide antigen of the kit can also be attached to a solid support.

In an additional embodiment, the invention includes a diagnostic kit for use in screening serum containing antigens of the mutant ErbB2 polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody can be a monoclonal antibody. The detecting means of the kit can include a second, labelled monoclonal antibody. Alternatively, or in addition, the detecting means can include a labelled, competing antigen.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

1. A naturally occurring cancer-associated mutant of a human ErbB2 polypeptide, comprising one or more mutations.

2. A mutant polypeptide according to claim 1 which is associated with NSCLC.

3. A mutant polypeptide according to claim 1, wherein the mutation is in the kinase domain of ErbB2.

4. A mutant polypeptide according to claim 1, wherein the mutation is an insertion.

5. A mutant polypeptide according to claim 1, wherein the mutation is an amino acid substitution.

6. A mutant polypeptide according to claim 4, wherein the insertion is selected from the group consisting of ins774(AYVM) and ins779(VGS).

7. A mutant polypeptide according to claim 5, wherein the amino acid substitution is selected from the group consisting of L755P, E914K and G776S.

8. A fragment of a mutant polypeptide according to claim 1, wherein said fragment comprises the mutation as described.

9. The complement of a nucleic acid selected from the group consisting of:
a nucleic acid encoding an ErbB2 polypeptide according to claim 1; a nucleic acid encoding an ErbB2 polypeptide according to claim 1, wherein the nucleic acid comprises one or more point mutations; a nucleic acid encoding an ErbB2 polypeptide according to claim 1, wherein the nucleic acid comprises one or more insertions; a nucleic acid encoding an ErbB2 polypeptide according to claim 1 which comprises one or more point mutations, wherein the point mutation occurs at one or more of positions 2263, 2704 and 2326 of ErbB2; a nucleic acid encoding an ErbB2 polypeptide according to claim 1, which comprises one or more point mutations, wherein the point mutation is HetTT2263/4CC, HetG2740A or HetG2326A; a nucleic acid encoding an ErbB2 polypeptide according to claim 1 which comprises one or more insertions, wherein the insertion occurs at one or more of positions 2322 or 2335 of ErbB2; and a nucleic acid encoding an ErbB2 polypeptide according to claim 1, which comprises one or more insertions, wherein the insertion is Het2322dup12 nt or Het2335ins9 nt.

10. A nucleic acid which hybridises specifically to a nucleic acid selected from the group consisting of: a nucleic acid encoding an ErbB2 polypeptide according to claim 1; a nucleic acid encoding an ErbB2 polypeptide according to claim 1, wherein the nucleic acid comprises one or more point mutations; a nucleic acid encoding an ErbB2 polypeptide according to claim 1, wherein the nucleic acid comprises one or more insertions; a nucleic acid encoding an ErbB2 polypeptide according to claim 1 which comprises one or more point mutations, wherein the point mutation occurs at one or more of positions 2263, 2704 and 2326 of ErbB2; a nucleic acid encoding an ErbB2 polypeptide according to claim 1, which comprises one or more point mutations, wherein the point mutation is HetTT2263/4CC, HetG2740A or HetG2326A; a nucleic acid encoding an ErbB2 polypeptide according to claim 1 which comprises one or more insertions, wherein the insertion occurs at one or more of positions 2322 or 2335 of ErbB2; and a nucleic acid encoding an ErbB2 polypeptide according to claim 1, which comprises one or more insertions, wherein the insertion is Het2322dup12 nt or Het2335ins9 nt.

11. A nucleic acid primer which directs specific amplification of a nucleic acid selected from the group consisting of: a nucleic acid encoding an ErbB2 polypeptide according to claim 1; a nucleic acid encoding an ErbB2 polypeptide according to claim 1, wherein the nucleic acid comprises one or more point mutations; a nucleic acid encoding an ErbB2 polypeptide according to claim 1, wherein the nucleic acid comprises one or more insertions; a nucleic acid encoding an ErbB2 polypeptide according to claim 1 which comprises one or more point mutations, wherein the point mutation occurs at one or more of positions 2263, 2704 and 2326 of ErbB2; a nucleic acid encoding an ErbB2 polypeptide according to claim 1, which comprises one or more point mutations, wherein the point mutation is HetTT2263/4CC, HetG2740A or HetG2326A; a nucleic acid encoding an ErbB2 polypeptide according to claim 1 which comprises one or more insertions, wherein the insertion occurs at one or more of positions 2322 or 2335 of ErbB2; and a nucleic acid encoding an ErbB2 polypeptide according to claim 1, which comprises one or more insertions, wherein the insertion is Het2322dup12 nt or Het2335ins9 nt.

12. A ligand which binds selectively to a polypeptide according to claim 1.

13. A ligand according to claim 12 which is an immunoglobulin.

14. A ligand according to claim 12, which is an antibody or an antigen-binding fragment thereof.

15. A method for the detection of oncogenic mutations, comprising the steps of:
(a) isolating a sample of naturally-occurring cellular material from a human subject;
(b) examining nucleic acid material from at least part of one or more ErbB2 genes in said cellular material; and
(c) determining whether such nucleic acid material comprises one or more mutations in a sequence encoding an ErbB2 polypeptide; or
(d) isolating a first sample of cellular material from a naturally-occurring tissue of a subject which is suspected to be cancerous, and a second sample of cellular material from a non-cancerous tissue of the same subject;
(e) examining nucleic acid material from at least part of one or more ErbB2 genes in both said samples of cellular material; and
(f) determining whether such nucleic acid material comprises one or more mutations in a sequence encoding an ErbB2 polypeptide; and said mutation being present in the naturally-occurring cellular material from the suspected cancerous tissue but not present in the cellular material from the non-cancerous tissue.

16. A method according to claim 15, wherein the mutation is a point mutation and occurs at occurs at one or more of positions 2263, 2704 and 2326 of ErbB2; or is an insertion, and occurs at one or more of positions 2322 or 2335 of ErbB2.

17. A method according to claim 16, wherein the mutation is a point mutation and is HetTT2263/4CC, HetG2740A or HetG2326A; or the mutation is an insertion, and is Het2322dup12 nt or Het2335ins9 nt.

18. A method for the detection of oncogenic mutations, comprising the steps of
(a) obtaining a sample of cellular material from a subject;
(b) screening said sample with a ligand according to claim 12; and
(c) detecting one or more mutant ErbB2 polypeptides in said sample.

19. A method according to claim 18, wherein the mutant ErbB2 polypeptide is a naturally occurring cancer-associated mutant of a human ErbB2 polypeptide, comprising one or more mutations.

20. Apparatus for detecting a mutation at a target sequence position in a nucleic acid encoding an ErbB2 polypeptide, comprising:
a sequence detecting device operable to monitor the sequence a sample of an amplification product of the nucleic acid to provide a sample data set specifying measured base pair identification data in a target domain extending from a start sequence position to an end sequence position; and
a data analysis unit connected to receive the sample data set from the sequencing device and operable to determine presence or absence of the mutation in the sample conditional on whether the measured base pair identification datum for the target sequence position corresponds to a reference base pair datum for the target sequence position.

21. The apparatus of claim 20, further comprising an output device operable to generate an output indicating the presence or absence of the mutation in the sample determined by the data analysis unit.

22. The apparatus of claim 21, wherein the output device comprises at least one of: a graphical user interface; an audible user interface; a printer; a computer readable storage medium; and a computer interpretable carrier medium.

23. An automated method for detecting a mutation at a target sequence position in a nucleic acid encoding an ErbB2 polypeptide, comprising:
sequencing a sample of an amplification product of the nucleic acid to provide a sample data set specifying measured base pair identification data in a target domain extending from a start sequence position to an end sequence position;
determining presence or absence of the mutation in the sample conditional on whether the measured base pair identification datum for the target sequence position corresponds to a reference base pair datum for the target sequence position; and
generating an output indicating the presence or absence of the mutation in the sample as established by the determining step.

24. Apparatus for detecting a mutation in an ErbB2 polypeptide, comprising:
a protein marking device loaded with a marker and operable to apply a marker to one or more target amino acids in a sample of the ErbB2 polypeptide; and
a marker reading device operable to determine presence or absence of the marker in the sample, thereby to indicate presence or absence of the mutation in the sample.

25. The apparatus of claim 24, wherein the marker comprises a ligand that binds preferentially to an ErbB2 polypeptide bearing the mutation.

26. The apparatus of claim 24, wherein the marker comprises a ligand that binds preferentially to an ErbB2 polypeptide of a wild-type without the mutation.

27. The apparatus of claim 24, wherein the marker is an antibody.

28. The apparatus of claim 27, wherein the protein marking device is configured to implement an ELISA process.

29. The apparatus of claim 24, wherein the protein marking device comprises a microarrayer.

30. The apparatus of claim 24, wherein the marker reading device is configured to read the sample optically.

31. The apparatus of claim 24, comprising an output device operable to generate an output indicating the presence or absence of the mutation in the sample as determined by the marker reading device.

32. The apparatus of claim 31, wherein the output device comprises at least one of: a graphical user interface; an audible user interface; a printer; a computer readable storage medium; and a computer interpretable carrier medium.

33. An automated method for detecting a mutation in an ErbB2 polypeptide, comprising:
applying a marker to one or more target amino acids in a sample of the ErbB2 polypeptide;
reading the sample after applying the marker to determine presence or absence of the marker in the sample, thereby to indicate presence or absence of the mutation in the sample; and
generating an output indicating the presence or absence of the mutation in the sample as determined by the reading step.

34. An apparatus or method according to claim 20, wherein the mutation is selected from the group consisting of a point mutation at one or more of positions 2263, 2704 and 2326 of ErbB2; a point mutation which is HetTT2263/4CC, HetG2740A or HetG2326A; an insertion, wherein the insertion occurs at one or more of positions 2322 or 2335 of ErbB2; and an insertion, wherein the insertion is Het2322dup12 nt or Het2335ins9 nt.

35. An apparatus or method according to claim 23, wherein the mutation is selected from the group consisting of a point mutation at one or more of positions 2263, 2704 and 2326 of ErbB2; a point mutation which is HetTT2263/4CC, HetG2740A or HetG2326A; an insertion, wherein the insertion occurs at one or more of positions 2322 or 2335 of ErbB2; and an insertion, wherein the insertion is Het2322dup12 nt or Het2335ins9 nt.

36. An apparatus or method according to claim 24, wherein the mutation is selected from the group consisting of a point mutation at one or more of positions 2263, 2704 and 2326 of ErbB2; a point mutation which is HetTT2263/4CC, HetG2740A or HetG2326A; an insertion, wherein the insertion occurs at one or more of positions 2322 or 2335 of ErbB2; and an insertion, wherein the insertion is Het2322dup12 nt or Het2335ins9 nt.

37. An apparatus or method according to claim 33, wherein the mutation is selected from the group consisting of a point mutation at one or more of positions 2263, 2704 and 2326 of ErbB2; a point mutation which is HetTT2263/4CC, HetG2740A or HetG2326A; an insertion, wherein the insertion occurs at one or more of positions 2322 or 2335 of ErbB2; and an insertion, wherein the insertion is Het2322dup12 nt or Het2335ins9 nt.

38. A method for identifying one or more compounds having anti-proliferative activity, comprising the steps of:
(a) providing one or more mutant ErbB2 polypeptides according to claim 1;
(b) contacting said polypeptide(s) with one or more compounds to be tested; and
(c) detecting an interaction between said one or more compounds and said mutant polypeptides.

39. A method according to claim 38, wherein the interaction is a binding interaction.

40. An assay for identifying one or more compounds having anti-proliferative activity, comprising the steps of:
(a) providing one or more mutant ErbB2 polypeptides in accordance with claim 1;
(b) providing a downstream substrate for the ErbB2 polypeptide;
(c) detecting modification of the substrate in presence of the compound(s) to be tested.

41. An assay according to claim 40, wherein the substrate modification is detected directly.

42. An assay according to claim 40, wherein the substrate is an enzyme which modifies a second substrate, which second modification is detectable.

43. A method or assay according to claim 38, wherein a reference level is determined for the assay in absence of the compound or compounds to be tested.

44. A method or assay according to claim 40, wherein a reference level is determined for the assay in absence of the compound or compounds to be tested.

45. A method for determining whether a patient is expected to be responsive to anti-ErbB2 therapy, comprising the steps of:
(a) isolating a sample of naturally-occurring cellular material from a human subject;
(b) examining nucleic acid material from at least part of one or more ErbB2 genes in said cellular material; and
(c) determining whether such nucleic acid material comprises one or more mutations in a sequence encoding an ErbB2 polypeptide.

46. A method for determining whether a patient is expected to be responsive to anti-ErbB2 therapy, comprising the steps of
(a) obtaining a sample of cellular material from a subject;
(b) screening said sample with a ligand according to the invention; and
(c) detecting one or more mutant ErbB2 polypeptides in said sample.

47. A method for treating a patient suffering from a tumour, comprising the steps of:
(a) determining if the tumour is ErbB2-dependent; and
(b) treating patients having ErbB2 dependent tumours with an inhibitor of ErbB2 activity.

48. A method for determining whether a patient is susceptible to therapy with Herceptin® or Omnitarg®, comprising the steps of:
(a) determining whether the patient is suffering from an ErbB2 dependent tumour; and
(b) administering Herceptin® and/or Omnitarg® to patients suffering from ErbB2 dependent tumours.

49. A method for determining whether a tumour is ErB2 dependent, comprising examining nucleic acid material from said tumour to identify the presence of any one or more mutations in the ErbB2 gene.
